SEQUENCE LISTING (5 sequences)

SEQ ID NO: 1 - DNA, 21 bp, synthetic construct
  source 1..21: mol_type = other DNA
tggtattcctcccgtggcttc

SEQ ID NO: 2 - DNA, 22 bp, synthetic construct
  source 1..22: mol_type = other DNA
ccccagtagacgcaatatacgc

SEQ ID NO: 3 - DNA, 20 bp, synthetic construct
  source 1..20: mol_type = other DNA
atagatgaacatgcgtctgg

SEQ ID NO: 4 - DNA, 20 bp, synthetic construct
  source 1..20: mol_type = other DNA
caaaatcctcatcaacaccg

SEQ ID NO: 5 - DNA, 23 bp, synthetic construct
  source 1..23: mol_type = other DNA
ctgaaagcttatctctgcgtggt

(19) United States
(12) Patent Application Publication
(10) Pub. No.: US 2023/0250497 A1
(43) Pub. Date: Aug. 10, 2023

(54) ONE-STEP NESTED PCR PRIMERS SET AND KIT MODIFIED WITH LOCKED NUCLEIC ACID FOR DETECTING AFRICAN SWINE FEVER VIRUS

(71) Applicant: Institute of Animal Health, Guangdong Academy of Agricultural Sciences, Guangzhou (CN)

(72) Inventors: Chunling Li, Guangzhou (CN); Keda Shi, Guangzhou (CN); Yan Li, Guangzhou (CN); Shaolun Zhai, Guangzhou (CN); Zhibiao Bian, Guangzhou (CN); Hongchao Gou, Guangzhou (CN); Kunli Zhang, Guangzhou (CN); Zhiyong Jiang, Guangzhou (CN); Pinpin Chu, Guangzhou (CN); Dongxia Yang, Guangzhou (CN)

(21) Appl. No.: 18/153,323

(22) Filed: Jan. 11, 2023

(30) Foreign Application Priority Data
Feb. 9, 2022 (CN) 202210121406X

(51) Int. Cl. C12Q 1/70; C12Q 1/6853

(52) U.S. Cl. CPC C12Q 1/701; C12Q 1/6853

(57) ABSTRACT

A one-step nested PCR primers set and a kit modified with locked nucleic acid for detecting African swine fever virus are provided, relating to the field of molecular biology. It includes an outer primer pair, an inner primer pair and a probe. Upstream and downstream primer sequences of the outer primer pair are shown in SEQ ID NO: 1 and SEQ ID NO: 2 respectively. Upstream and downstream primer sequences are shown in SEQ ID NO: 3 and SEQ ID NO: 4 respectively. A sequence of the probe is shown in SEQ ID NO: 5. Based on the principle of nested PCR, the Tm value and stability of outer primers are improved by designing two pairs of nested primers and simultaneously modifying the outer primers with locked nucleic acid, and two independent circular nested amplification are performed, which has high sensitivity and specificity, easiness in operation and less cross contamination.

### STATEMENT REGARDING SEQUENCE LISTING

The sequence listing associated with this application is provided in text format in lieu of a paper copy and is hereby incorporated by reference into the specification. The name of the XML file containing the sequence listing is 22132TBYX-USP1-SL.xml. The XML file is 5,359 bytes; is created on Jan. 11, 2023; and is being submitted electronically via EFS-Web.

### TECHNICAL FIELD

The disclosure relates to the field of molecular biology, and more particularly to a one-step nested polymerase chain reaction (PCR) primers set and a kit modified with locked nucleic acid for detecting African swine fever virus.

### BACKGROUND

African swine fever (ASF) is an acute and virulent infectious disease caused by African swine fever virus (ASFV) infecting domestic pigs and various wild boars. It has a rapid incidence and a mortality rate of 90%-100%, which has seriously endangered the pig industry in China and is classified as a Class I infectious disease by the Ministry of Agriculture of China. ASFV belongs to the double-stranded DNA virus order ASFV family, ASFV genus. ASFV is a large individual with a complex structure, its surface is an icosahedral structure and a layer of lipid-containing capsule. Its genome encodes a large number of proteins, which are easy to mutate, and has a variety of natural host genotypes. ASFV has a strong resistance to the outside world and has a long survival time in the environment. It is reported that it can survive in blood, feces and tissues for up to half a year, in infected raw or incompletely cooked pork products for up to three months, and in frozen meat for several years. Moreover, ASFV has many genotypes and is easy to mutate. The ASF vaccine in China is in the research and development stage, but it will take some time to develop and apply it successfully. At present, the main means to prevent and control ASF is still to monitor and eliminate the source. Therefore, prevention and control rely on early diagnosis and detection with high sensitivity and specificity to eliminate the source, so that laboratory diagnosis is particularly important.

At present, the laboratory diagnostic methods for ASFV mainly include antibody detection by fluorescence immunoassay, enzyme-linked immunosorbent assay (ELISA) antibody detection method, nucleic acid detection by fluorescence quantitative polymerase chain reaction (PCR) method and conventional PCR method. The fluorescence quantitative PCR and the conventional PCR are one of the routine and rapid detection methods in the laboratory. The detection limit is approximately 200 to 1000 copies/reaction. However, there is a time difference between the collection of samples from pig farms and laboratory tests, which leads to nucleic acid degradation; or poor sample collection quality, improper nucleic acid extraction, or different sample types and disease courses, which may lead to the viral load of the test samples being lower than the detection limit of traditional fluorescence PCR, leading to false-negative results. Digital PCR can be used to detect the trace viral load in samples, but it often requires independent supporting reagents, equipment and software, as well as professional personnel, and is expensive.

### SUMMARY

The purpose of the disclosure is to provide a one-step nested polymerase chain reaction (PCR) primers set and a kit modified with locked nucleic acid for detecting African swine fever virus (ASFV), so as to solve the problems existing in the prior art. Based on the principle of nested PCR, two pairs of nested primers are designed in the disclosure, outer primers are subjected to locked nucleic acid modification simultaneously, so that the Tm value and the stability of the outer primers are improved, and two times of independent circular nested amplification are performed; and the method has the advantages of higher sensitivity and specificity, easiness in operation and less cross contamination.

To achieve the above purpose, the disclosure provides the following solutions.

The disclosure provides a one-step nested polymerase chain reaction (PCR primers set modified with locked nucleic acid for detecting ASFV, including an outer primer pair, an inner primer pair and a probe. A sequence of an upstream primer of the outer primer pair is shown in SEQ ID NO: 1, and a sequence of a downstream primer of the outer primer pair is shown in SEQ ID NO: 2. A sequence of an upstream primer of the inner primer pair is shown in SEQ ID NO: 3, and a sequence of a downstream primer of the inner primer pair is shown in SEQ ID NO: 4. Bases at second, ninth, thirteenth, sixteenth and eighteenth positions in the sequence of the upstream primer of the outer primer pair are the locked nucleic acid; and bases at first, seventh, tenth, thirteenth and seventeenth positions in the sequence of the downstream primer of the outer primer pair are the locked nucleic acid. A sequence of the probe is shown in SEQ ID NO: 5.

The disclosure also provides an application of the one-step nested PCR primers set modified with locked nucleic acid for detecting ASFV in preparing a kit for detecting ASFV.

The disclosure also provides a kit for detecting ASFV, which includes the one-step nested PCR primers set modified with locked nucleic acid for detecting ASFV.

In one embodiment, a concentration of the probe in a PCR reaction system of the kit is 100 nanomoles per liter (nmol/L).

In one embodiment, the kit further includes Probe Master Mix and nuclease-free water.

In one embodiment, an annealing temperature of the outer primer pair in a PCR reaction system of the kit is 67 Celsius degree (° C.). Specifically, the Probe Master Mix is AceQ® Universal U+ Probe Master Mix V2 purchased from Vazyme Biotech Co., Ltd.

In one embodiment, an annealing temperature of the inner primer pair in a PCR reaction system of the kit is 59° C.

In one embodiment, concentrations of the upstream primer and the downstream primer of the inner primer pair in a PCR reaction system of the kit is 300 nmol/L.

The disclosure discloses technical effects as follows.

The detection limit of the one-step nested fluorescence PCR method modified with nucleic acid of the disclosure for ASFV is 100 times higher than the minimum detection limit of the conventional probe fluorescence quantitative PCR and has good sensitivity.

The intra-group coefficient of variation of the one-step nested fluorescence PCR method modified with locked nucleic acid of the disclosure is less than 1%, and the coefficient of variation among groups is less than 3%, indicating that the method has good repeatability.

The one-step nested fluorescence PCR method modified with locked nucleic acid of the disclosure has higher positive detection rate of nucleic acid in clinical samples than that of conventional probe fluorescence quantitative PCR, and higher sensitivity.

### BRIEF DESCRIPTION OF DRAWINGS

In order to describe embodiments or technical solutions of the disclosure more clearly, the following will briefly introduce drawings required in the embodiments. Apparently, the drawings in the following description are only some embodiments of the disclosure. For those skilled in the related art, they can also obtain other drawings based on these drawings without paying creative labor.

FIG. 1 illustrates a schematic diagram of design of nested primers modified with locked nucleic acid and a probe for one-step fluorescence PCR method.

FIG. 2A illustrates gel imaging of gradient annealing temperature of outer primers.

FIG. 2B illustrates gel imaging of gradient annealing temperature of inner primers.

FIG. 3A illustrates amplification curves of inner primers and probes with concentration of 200-600 nmol/L.

FIG. 3B illustrates amplification curves of inner primers and probes with concentration of 100-500 nmol/L.

FIG. 4A illustrates fluorescent amplification curves of the one-step nested fluorescence PCR modified with locked nucleic acid of standard plasmid of African swine fever virus (ASFV), where 1-5 are respectively corresponds to 3×10⁴, 3×10³, 3×10², 3×10¹, 3×10⁰ copies/reaction, and NC represents negative control.

FIG. 4B illustrates fluorescence PCR amplification curves of common probes of the standard plasmid of ASFV, where 1˜4 are respectively corresponds to 3×10⁴, 3×10³, 3×10², 3×10¹, 3×10⁰ copies/reaction, and NC represents negative control.

FIG. 4C illustrates standard curves of one-step nested fluorescence PCR modified with locked nucleic acid and common probe fluorescence PCR of the standard plasmid of ASFV.

### DETAILED DESCRIPTION OF EMBODIMENTS

A variety of exemplary embodiments of the disclosure are described in detail. The detailed description should not be considered as a limitation of the disclosure, but should be understood as a more detailed description of some aspects, features and embodiments of the disclosure.

It should be understood that the terms used herein is for the purpose of describing particular embodiments only and is not intended to be limiting of the disclosure. In addition, for numerical ranges in the disclosure, it should be understood that each intermediate value between upper and lower limits of the range is also specifically disclosed. Intermediate values within any stated value or stated range, and each smaller range between any other stated value or intermediate values within the stated range are also included in the disclosure. The upper and lower limits of these smaller ranges may be independently included or excluded from the range.

Unless otherwise stated, all technical and scientific terms used herein have the same meanings as those commonly understood by those skilled in the art described herein. Although the disclosure only describes preferred methods and materials, any methods and materials similar or equivalent to those described herein can be used in the implementation or testing of the disclosure. All documents mentioned in this specification are incorporated by reference to disclose and describe methods and/or materials related to the documents. In case of conflict with any incorporated literature, the contents of this manual shall prevail.

Without departing from the scope or spirit of the disclosure, it will be apparent to those skilled in the art that various improvements and changes can be made to the specific embodiments of the specification of the disclosure. Other embodiments obtained from the specification of the disclosure will be apparent to those skilled in the art. The description and embodiments of the disclosure are only illustrative.

As used herein, the terms “comprising”, “including”, “having”, “containing”, and the like are open-ended terms that mean including but not limited to.

### Embodiment 1

### Primer and Probe Design

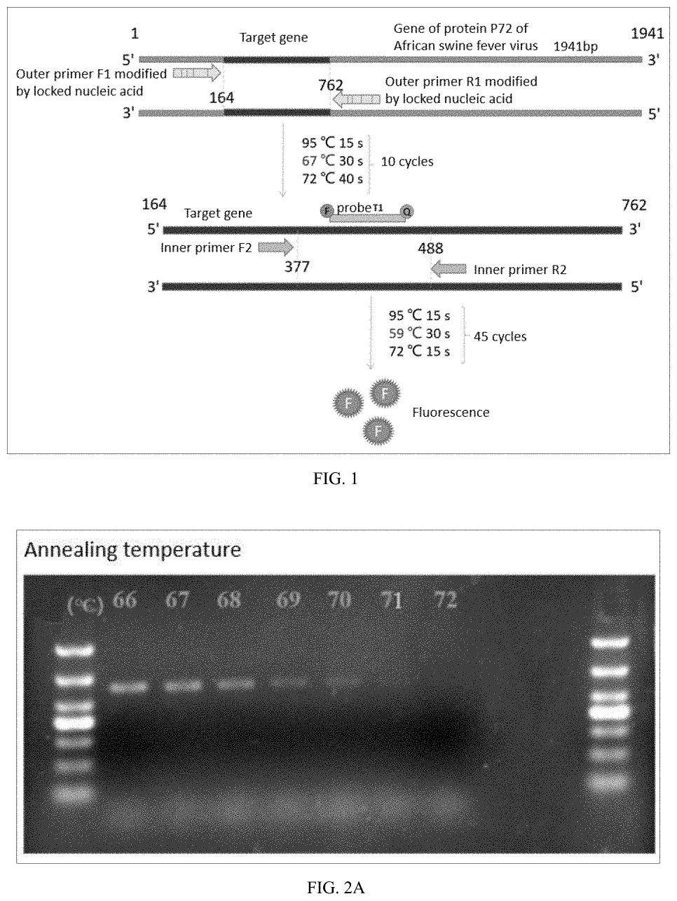

Referring to the nucleic acid sequence (Gene ID: 22220311) of the whole-genome nucleic acid sequence of protein B646L (P72) of African swine fever virus (ASFV), a total length of the gene sequence is 1941 base pairs (bp). After sequence alignment of mutant strains, conserved sequences are selected, and nested primers and probes are designed through Oligo 7 as primers modified with locked nucleic acid and a probe for one-step nested fluorescence PCR, as shown in FIG. 1. Nucleic acid sequences of nested primers and a probe are shown in Table 1

TABLE 1
Base sequences of one-step nested PCR primers and probe modified
with locked nucleic acid

| | Nucleic acid sequence (5′-3′) |
|---|---|
| Outer primer F1 | TG(+)GTATTCC(+)TCCC(+)GTG(+)GC(+)TTC (SEQ ID NO: 1) |
| Outer primer R1 | C(+)CCCAGT(+)AGA(+)CGC(+)AATA(+)TACGC (SEQ ID NO: 2) |
| Inner primer F2 | ATAGATGAACATGCGTCTGG (SEQ ID NO: 3) |
| Inner primer R2 | CAAAATCCTCATCAACACCG (SEQ ID NO: 4) |
| Probe T1 | FAM-CTGAAAGCTTATCTCTGCGTGGT-BHQ1 (SEQ ID NO: 5) |

Note:
(+) represents the base of the nucleic acid modified with locked nucleic acid

Procedures of the one-step nested fluorescence PCR method modified with locked nucleic acid is as shown in FIG. 1. In the first step, the outer primers F1 and R1 modified with locked nucleic acid are used to amplify at sites 164˜762 of the sequence, and a 592 bp product fragment is obtained after 10 cycles. In the second step, based on the product fragment amplified in the first step. the inner primers F2 and R2 are used to amplify at sites 377-488 of the sequence, and combined with the probe simultaneously to release carboxyfluorescein (FAM) fluorescent groups for the fluorescence PCR equipment to collect signals.

### Embodiment 2

### Optimization of Amplification Conditions

1. Annealing Temperature of Outer Primers

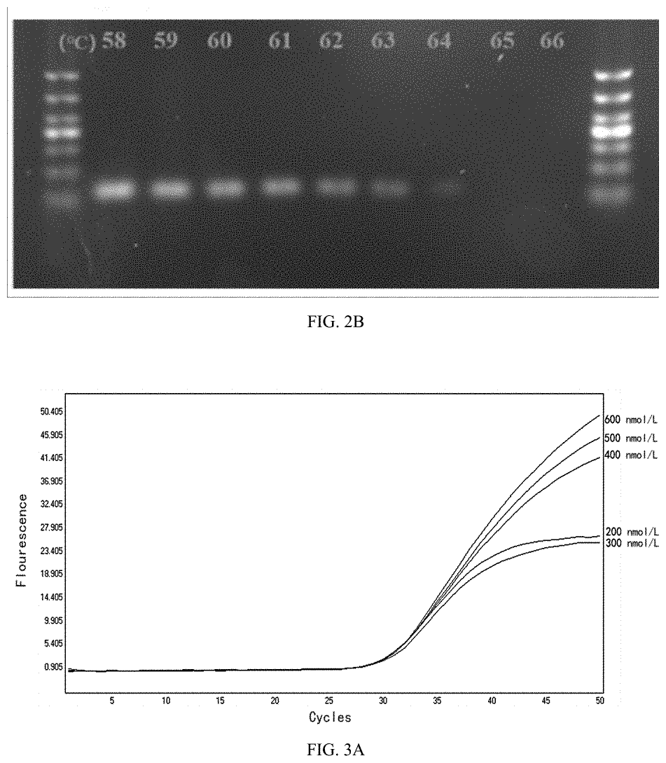

The outer primers F1 and R1 are used for conventional PCR amplification. The annealing temperature is set at 66˜72° C., and the gradient is 1° C., and other conditions are the same as those of conventional PCR. The DNA template added is standard plasmid of the protein B646L (P72) of ASFV. After reaction, PCR amplification products are taken and detected by agarose gel electrophoresis. As shown in FIG. 2A, the optimum annealing temperature is 67° C.

2. Annealing Temperature of Inner Primers

The inner primers F2 and R2 are used for conventional PCR amplification. The annealing temperature is set at 58-66° C. and the gradient is 1° C., and other conditions are the same as those of conventional PCR. The DNA template added is the standard plasmid of the protein B646L (P72) of ASFV. After reaction, PCR amplification products are taken and detected by agarose gel electrophoresis. As shown in FIG. 2B, the optimum annealing temperature is 59° C.

3. Optimization of Inner Primer Concentration

The optimum annealing temperature is used for one-step nested fluorescence PCR modified with locked nucleic acid. The reaction conditions are: pre-denatured at 95° C. for 5 minutes; denatured at 95° C. for 15 seconds, annealed at 67° C. for 30 seconds, and extended at 72° C. for 40 seconds, with 10 cycles; denatured at 95° C. for 15 seconds, annealed at 59° C. for 30 seconds, and extended at 72° C. for 15 seconds, with 45 cycles. The concentrations of the inner primers are set as 200 nanomoles per liter (nmol/L), 300 nmol/L, 400 nmol/L, 500 nmol/L and

600 nmol/L respectively, and a reaction system is as follows:
2 micromoles per liter (μM) outer primer F1, 0.5 μL,
2 μM outer primer R1, 0.5 μL,
10 μM inner primer F2, 0.4 μL˜1.2 μL,
10 μM inner primer R2, 0.4 μL˜1.2 μL,
10 μM probe T1, 0.2 μL,

### Probe Master Mix, 10 μL,

ASFV plasmid 5 μL, and
residual nuclease-free water to make up the total volume to be 20 μL.

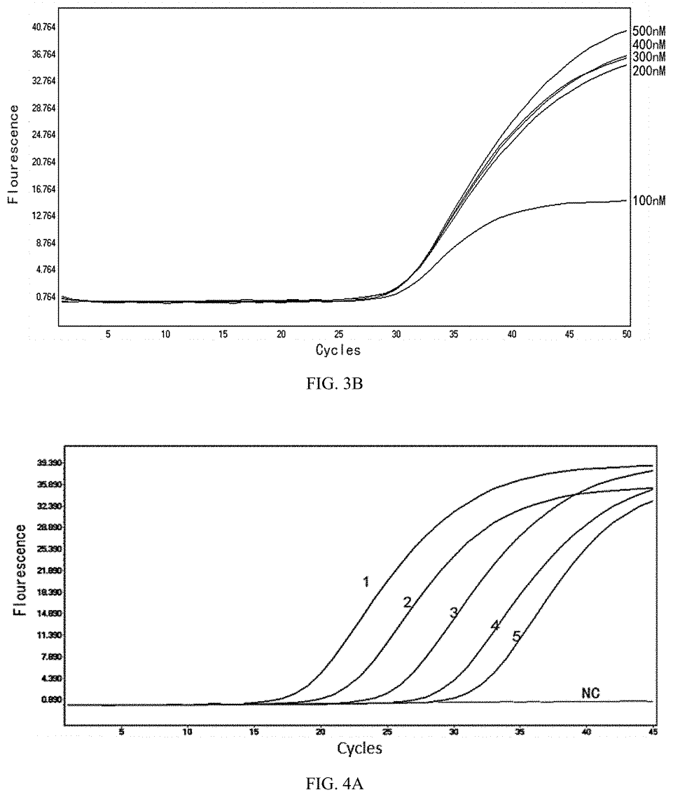

The results are as shown in FIG. 3A, the optimum concentrations of the inner primer F2 and the inner primer R2 are 300 nmol/L.

4. Probe Concentration Optimization

The optimum annealing temperature is used for one-step nested fluorescence PCR modified with locked nucleic acid. The reaction conditions ae: pre-denatured at 95° C. for 5 minutes; denatured at 95° C. for 15 seconds, annealed at 67° C. for 30 seconds, and extended at 72° C. for 40 seconds, with 10 cycles; denatured at 95° C. for 15 seconds, annealed at 59° C. for 30 seconds, and extended at 72° C. for 15 seconds, with 45 cycles. The concentrations of the probe T1 are set to 100 nmol/L, 200 nmol/L, 300 nmol/L, 400 nmol/L and 500 nmol/L. A reaction system is as follows:

2 μM outer primer F1, 0.5 μL,
2 μM outer primer R1, 0.5 μL,
10 μM inner primer F2, 0.6 μL,
10 μM inner primer R2, 0.6 μL,
10 μM probe T1, 0.2 μL˜1.0 μL,

### Probe Master Mix, 10 μL,

standard plasmid of ASF, 5 μL, and
residual nuclease water to make up the total volume to be 20 μL.

As shown in FIG. 3B, the optimum concentration of the probe T1 is 100 nmol/L.

### Embodiment 3

### Establishment of Standard Curve

The standard plasmid of ASFV P72 is subjected to 10-fold gradient dilution according to the copies/reaction of 3×10⁴, 3×10³, 3×10², 3×10¹, 3×10⁰, nuclease-free water is used as negative control, one-step nested fluorescence PCR modified with locked nucleic acid is performed according to the optimized reaction system and conditions in the embodiment 2, to obtain amplification curves of plasmids with different concentrations, and standard curves are drawn. The common probe fluorescence PCR is used as the control.

Judgment of detection results is as follows.

If the fluorescence quantitative PCR result shows that the Cq value is less than or equal to 31 and there is a specific “S” type amplification curve, it is determined as positive. If the fluorescence quantitative PCR result shows that the Cq value is greater than 31, or there is no specific “S” type amplification curve, it is determined as negative.

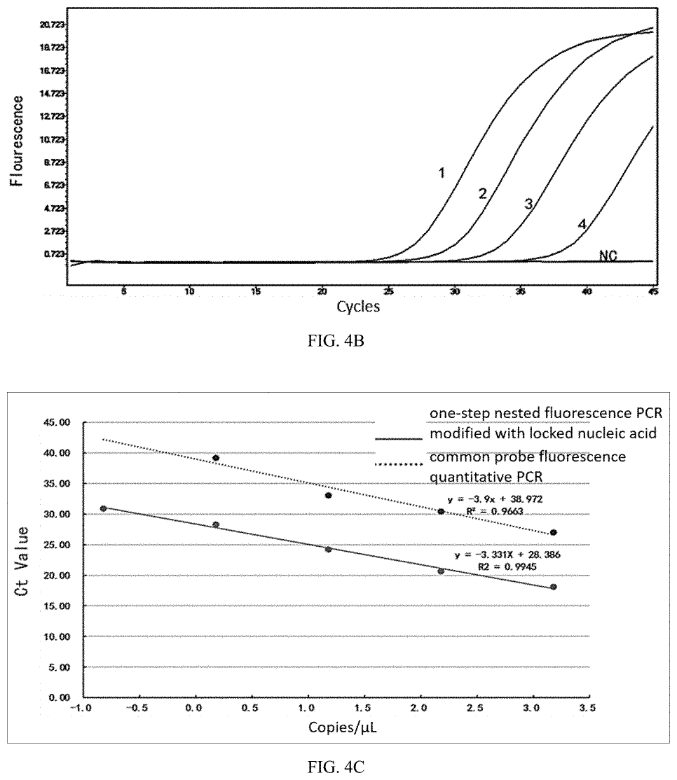

With respect to results, amplification curves of the one-step nested fluorescence PCR modified with locked nucleic acid are shown in FIG. 4A; fluorescence amplification curves of common probe fluorescence PCR are shown in FIG. 4B; and results of the one-step nested fluorescence PCR modified with locked nucleic acid and the common probe fluorescence PCR are shown in FIG. 4C. The results show that the minimum detection limit of the one-step nested fluorescence PCR modified with locked nucleic acid is 3×10⁰ copies/reaction, and the minimum detection limit of the common probe fluorescence PCR is 3×10² copies/reaction. The minimum detection limit of one-step nested fluorescence PCR modified with locked nucleic acid is 100 times higher than that of the common probe fluorescence PCR.

### Embodiment 4

### Repeatability Test of One-Step Nested Fluorescence PCR Modified with Locked Nucleic Acid

The standard plasmid of ASFV P72 is subjected to 10-fold gradient dilution according to the copies/reaction of 3×10⁴, 3×10³, 3×10², 3×10¹, 3×10⁰ , three independent replicates are performed, with three replicates for each concentration, and one-step nested fluorescence PCR modified with locked nucleic acid is performed according to the optimized reaction system and conditions in the embodiment 2.

With respect to results, as shown in Table 2, the intra-group coefficient of variation is less than 1%, and the coefficient of variation among groups is less than 3%. It shows that the established method has good repeatability and reliable results.

TABLE 2
Repeatability test of one-step nested fluorescence
PCR modified with locked nucleic acid

| | intra-group | | among groups | |
|---|---|---|---|---|
| Plasmid | | Coefficient of | | Coefficient of |
| (Copies/μL) | x̅ ± s | variation (%) | x̅ ± s | variation (%) |
| 1.5 × 10³ | 18.56 ± 0.12 | 0.62 | 18.79 ± 0.46 | 2.45 |
| 1.5 × 10² | 21.51 ± 0.10 | 0.48 | 21.67 ± 0.64 | 2.95 |
| 1.5 × 10¹ | 24.84 ± 0.05 | 0.21 | 24.97 ± 0.48 | 1.90 |
| 1.5 × 10⁰ | 27.83 ± 0.19 | 0.67 | 28.21 ± 0.14 | 0.50 |
| 1.5 × 10⁻¹ | 30.08 ± 0.06 | 0.21 | 30.66 ± 0.77 | 2.50 |

### Embodiment 5

### Detection of Clinical Samples by One-Step Nested Fluorescence PCR Modified with Locked Nucleic Acid

A total of 96 sample nucleic acids suspected to be clinically suffering from ASF are selected for detection, and the clinical sample nucleic acids are extracted from the pig ward of the Institute of Animal Health, Guangdong Academy of Agricultural Sciences. The method of the disclosure is used for detection, the amplification primers and the probe are as shown in the embodiment 1, the reaction conditions and system are as shown in the embodiment 2, and the positive judgement is as shown in the embodiment 3. At the same time, the common probe fluorescence PCR is used for detection and parallel comparison.

With respect to results, the results of the one-step nested PCR modified with locked nucleic acid are 55 positive, 41 negative, with a positive detection rate of 57.3%; the results of the common probe fluorescence PCR are 47 positive and 49 negative, with a positive detection rate of 48.9%. It is concluded that the one-step nested PCR modified with locked nucleic acid is more sensitive than the common probe fluorescence PCR.

TABLE 3
Clinical detection of common probe method and one-step nested
fluorescence PCR modified with locked nucleic acid

| | One-step nested fluorescence | |
|---|---|---|
| | quantitative PCR modified | common |
| Detection of ASFV | with locked nucleic acid | probe PCR |
| Positive | 55 | 47 |
| Negative | 41 | 49 |
| Total | 96 | 96 |
| Positive detection rate | 57.3% | 48.9% |

The above-described embodiments only describe the preferred mode of the disclosure, do not limit the scope of the disclosure. On the premise of not deviating from the design spirit of the disclosure, all changes and improvements made by those skilled in the art to the technical solution of the disclosure should fall within the scope of protection defined in the claims of the disclosure.

What is claimed is:

1. A one-step nested polymerase chain reaction (PCR) primers set modified with locked nucleic acid for detecting African swine fever virus, comprising:
an outer primer pair, an inner primer pair and a probe;
wherein a sequence of an upstream primer of the outer primer pair is shown in SEQ ID NO: 1, and a sequence of a downstream primer of the outer primer pair is shown in SEQ ID NO: 2;
wherein a sequence of an upstream primer of the inner primer pair is shown in SEQ ID NO: 3, and a sequence of a downstream primer of the inner primer pair is shown in SEQ ID NO: 4;
wherein bases at second, ninth, thirteenth, sixteenth and eighteenth positions in the sequence of the upstream primer of the outer primer pair are the locked nucleic acids; and bases at first, seventh, tenth, thirteenth and seventeenth positions in the sequence of the downstream primer of the outer primer pair are the locked nucleic acids; and
wherein a sequence of the probe is shown in SEQ ID NO: 5.

2. A use of the one-step nested PCR primers set modified with locked nucleic acid for detecting African swine fever virus according to claim 1, comprising:
preparing a kit for detecting African swine fever virus by using the one-step nested PCR primers set modified with locked nucleic acid detecting African swine fever virus.

3. A kit for detecting African swine fever virus, comprising:
the one-step nested PCR primers set modified with locked nucleic acid for detecting African swine fever virus according to in claim 1.

4. The kit for detecting African swine fever virus according to claim 3, wherein a concentration of the probe in a PCR reaction system of the kit is 100 nanomoles per liter (nmol/L).

5. The kit for detecting African swine fever virus according to claim 3,further comprising Probe Master Mix and nuclease-free water.

6. The kit for detecting African swine fever virus according to claim 3, wherein an annealing temperature of the outer primer pair in a PCR reaction system of the kit is 67 Celsius degree (° C.).

7. The kit for detecting African swine fever virus according to claim 3, wherein an annealing temperature of the inner primer pair in a PCR reaction system of the kit is 59° C.

8. The kit for detecting African swine fever virus according to claim 3, wherein concentrations of the upstream primer and the downstream primer of the inner primer pair in a PCR reaction system of the kit is 300 nmol/L.
